(19) United States
(12) Patent Application Publication
(10) Pub. No.: US 2005/0038525 A1
(43) Pub. Date: Feb. 17, 2005

(54) Shock absorbing prosthetic foot for use with prosthetic ankle

(75) Inventors: Jeffrey L. Doddroe, Washington Court House, OH (US); Robert E. Arbogast, Mt. Sterling, OH (US); James M. Colvin, Hilliard, OH (US); James W. Capper, Washington Court House, OH (US)

Correspondence Address: STANDLEY LAW GROUP LLP, 495 METRO PLACE SOUTH, SUITE 210, DUBLIN, OH, 43017, US

(73) Assignee: The Ohio Willow Wood Company, Mt. Sterling, OH (US)

(21) Appl. No.: 10/868,518

(22) Filed: Jun. 15, 2004

Related U.S. Application Data
(63) Continuation-in-part of application No. 10/352,902, filed on Jan. 29, 2003.
Application No. 10/352,902 is a continuation of application No. 09/502,455, filed on Feb. 11, 2000, now Pat. No. 6,602,295.
(60) Provisional application No. 60/135,704, filed on May 24, 1999.

(51) Int. Cl. A61F 2/66; A61F 2/62

(52) U.S. Cl. 623/55; 623/38; 623/52

(57) ABSTRACT

A low profile prosthetic foot designed for use with a prosthetic ankle. The prosthetic foot includes at least one toe spring and at least one heel spring. The prosthetic foot is able to absorb shocks generated during ambulation of a user of said foot. When a prosthetic ankle is attached to the prosthetic foot, the position (height) of the ankle preferably approximates the position (height) of a typical human ankle.

The present application is a continuation-in-part of U.S. application Ser. No. 10/352,902, filed on Jan. 29, 2003, which is a continuation of U.S. application Ser. No. 09/502,455, filed on Feb. 11, 2000, and now U.S. Pat. No. 6,602,295, which is based on Provisional Application No. 60/135,704, filed on May 5, 1999.

### BACKGROUND AND SUMMARY OF THE INVENTION

The present invention is directed to a shock absorbing prosthetic foot that is able to absorb the shocks developed during ambulation with efficient energy transfer between heel strike and toe-off, while simultaneously enhancing stability. Certain embodiments of the shock absorbing foot of the present invention are designed for use with a prosthetic ankle, such as a multi-axis articulating prosthetic ankle.

Specialized prosthetic feet have recently been developed in an effort to satisfy the specialized needs of different amputees. For example, active amputees who engage in sports or other strenuous physical activities typically require a prosthetic foot which is capable of both absorbing energy during a heel strike of each step, of efficiently transferring the energy to the toe of the prosthesis as the step progresses, and of releasing the stored energy at the moment of toe-off to provide energy for the next step.

In particular, during ambulation the foot initially contacts the ground at the heel. During strenuous activities, it is desirable for a prosthesis to be able to absorb the shock of this heel strike, and to transfer the absorbed energy to the toe portion of the prosthetic foot for release upon the subsequent toe-off so that the rebound energy is maximized. An effort to design a prosthetic foot capable of storing and subsequently releasing energy during ambulation is disclosed in U.S. Pat. No. 5,458,656 in which the pylon is formed of two telescoping parts connected by a spring. An upper part of the pylon incorporates a top adapter for connection to the residual stump of the wearer while the bottom part supports heel and toe springs. The energy of a heel strike is absorbed by the spring during telescoping of the pylon and is intended to be released as the load is removed from the prosthesis during toe-off.

As can be seen from the above-referenced prosthetic foot, known energy transferring designs can be quite bulky and inefficient. Additionally, such prosthetic foot designs are not amenable to use with a prosthetic ankle due to their overall height. More specifically, the height of such a prosthetic foot makes it impossible to use a prosthetic ankle, both because the location of a prosthetic ankle attached to such a foot would be too high, and because many amputees will not have an adequate amount of space available below their residual limbs.

Therefore, as can be seen, it is desirable to provide a prosthetic foot which is not subject to the shortcomings of the prior art. Consequently, the present invention is directed to a prosthetic foot that can absorb energy on heel strike and efficiently transfer the energy to the toe of the prosthesis for use during toe-off. Such a prosthetic foot of the present invention is also afforded with enhanced stability, control, function and durability. Particular embodiments of the present invention are designed for use with a prosthetic ankle.

According to one embodiment of the present invention, a prosthetic foot includes an adapter element securable to a residual limb, a foot plate having a heel portion and a toe portion along the length thereof, at least one toe spring connected between the adapter element and the toe portion of the foot plate, and a heel spring connected between the adapter element and the heel portion.

Since the toe spring of the prosthetic foot according to this feature of the invention is connected between the foot plate and to the adapter element, it can efficiently transfer energy to the toe portion during toe-off.

According to another embodiment of the present invention, a prosthetic foot comprises an adapter element securable to a residual limb, a foot plate having a heel portion and a toe portion along the length thereof, and at least one toe spring connected between the adapter element and the toe portion of the foot plate, the toe spring comprising a curved leaf spring whose concave side exhibits a plurality of transverse ribs. The transverse ribs create a more constant stress spring, and effectively distribute the bending stresses along the length of the spring. This minimizes the risk of delamination of the toe spring and permits more efficient energy transfer during toe-off.

According to yet another embodiment of the present invention, a prosthetic foot comprises a tubular pylon having one end securable to a residual limb, a collar mounted to the pylon for movement along the length of the pylon, a toe spring extending from the collar, a heel spring extending from the collar such that the toe and heel springs comprise toe and heel portions of the prosthetic foot, a further heel spring connected between the heel portion and another end of the pylon, and a non-extensible band extending between the heel portion and the collar.

The further heel spring according to this embodiment of the present invention reduces loading at the socket of the prosthesis, and the non-extensible band limits the movement of the collar away from the heel during the rebound of the heel spring, thereby promoting efficient energy transfer to the toe spring for release during toe-off.

In an alternate embodiment of the present invention, a prosthetic foot comprises a tubular pylon having one end securable to a residual limb, a foot plate having a heel portion and a toe portion along the length thereof, a collar mounted to the pylon for movement along the length of the pylon, at least one toe spring connected between the collar and the toe portion of the foot plate, and a heel spring connected between the heel portion and another end of the pylon.

Other embodiments of a prosthetic foot of the present invention are designed for use with a prosthetic ankle having a connector portion that is securable to a residual limb. These embodiments of the prosthetic foot may include a foot plate having a heel portion and a toe portion along the length thereof, at least one toe spring connected between the prosthetic ankle and the toe portion of the foot plate, and a heel spring connected between the prosthetic ankle and the heel portion of the foot plate. Such embodiments of the prosthetic foot are generally of lower profile than the previously described exemplary embodiments to ensure that the prosthetic ankle is located appropriately with respect to the overall prosthetic limb.

Embodiments of a prosthetic foot of the present invention may make use of elements constructed from layers of precured composite sheets bonded together with adhesives.

### BRIEF DESCRIPTION OF THE DRAWINGS

In addition to the features mentioned above, other aspects of the present invention will be readily apparent from the following descriptions of the drawings and exemplary embodiments, wherein like reference numerals across the several views refer to identical or equivalent features, and wherein:

FIG. 1 is a side perspective view of one exemplary embodiment of a prosthetic foot of the present invention;

FIG. 1A is a top perspective view of a foot plate portion of the prosthetic foot illustrated in FIG. 1;

FIG. 2 is a side perspective view of an alternate exemplary embodiment of a prosthetic foot of the present invention;

FIG. 3 is a side view of another exemplary embodiment of a prosthetic foot of the present invention;

FIG. 4 is a side view of yet another exemplary embodiment of a prosthetic foot of the present invention;

FIG. 5 is an exploded view of an example of a top adapter usable with a prosthetic foot of the present invention;

FIGS. 6a-6c show a top, side and front view, respectively, of a low profile embodiment of a prosthetic foot according to the present invention, wherein a prosthetic ankle is attached thereto;

FIG. 7 is an enlarged version of the side view of FIG. 6b, wherein various previously hidden components are visible;

FIG. 8 is an assembly view of the prosthetic foot shown in FIG. 7;

FIGS. 9a-9c depict an alternate embodiment of the prosthetic foot of FIGS. 6-8, wherein an auxiliary toe spring is employed;

FIGS. 10a-10c show a top, side and front view, respectively, of a low profile embodiment of another prosthetic foot according to the present invention, wherein a prosthetic ankle is attached thereto;

FIGS. 11a-11c show a top, side and front view, respectively, of yet another embodiment of a low profile prosthetic foot according to the present invention, wherein a prosthetic ankle is attached thereto;

FIG. 12 is an enlarged version of the side view of FIG. 11b, wherein various previously hidden components are visible; and

FIG. 13 is an assembly view of the prosthetic foot shown in FIG. 12;

FIGS. 14a and 14b illustrate a top plan view and a side sectional view, respectively, of the prosthetic ankle shown in FIGS. 6-13.

### DETAILED DESCRIPTION OF THE EXEMPLARY EMBODIMENT(S)

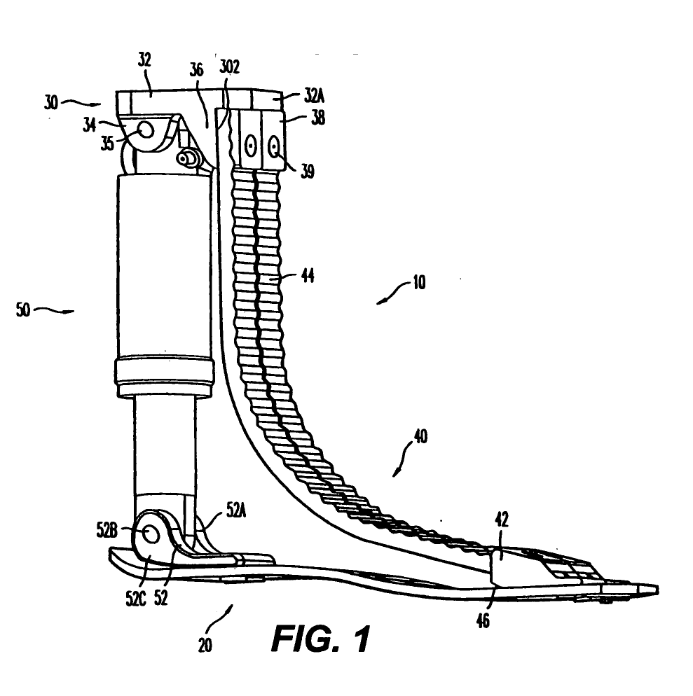

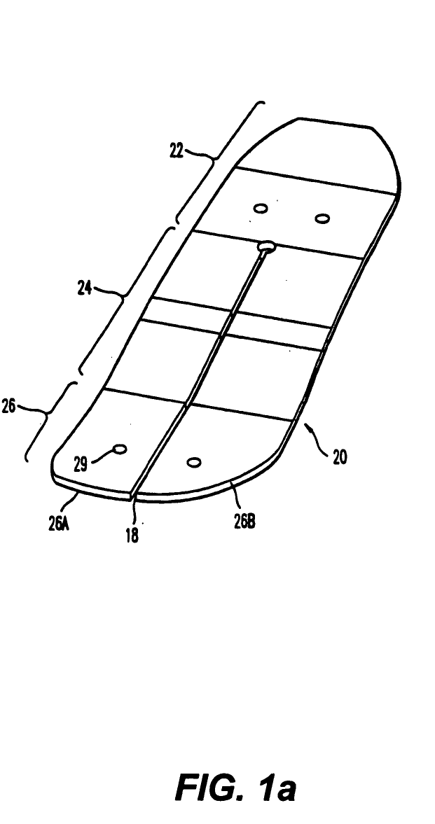

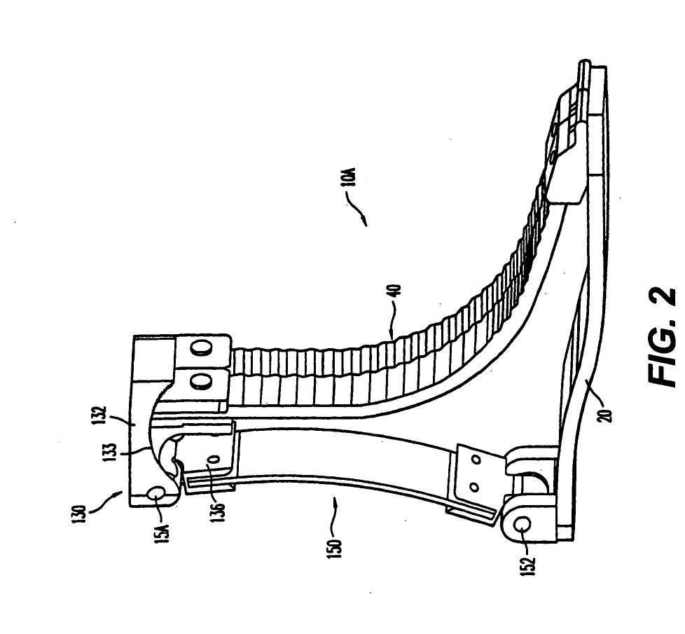

Referring now to the drawings wherein like reference numerals designate identical or corresponding parts throughout the several views, according to a first embodiment of the invention illustrated in the non-limiting FIGS. 1 and 2 of the drawings, and in particular the variant of FIG. 1, the main components of a prosthetic foot 10 comprise contoured foot plate 20, a top adapter 30, a pair of toe springs 40 and a heel spring/shock absorber 50.

The contoured foot plate 20 is generally conventional and could be that disclosed in U.S. Pat. No. 4,865,612, except as set forth below. It includes a heel portion 22, an arch portion 24 and a ball portion 26 (FIG. 1a). A slot 28 may extend through the foot plate and longitudinally from the toe portion to the heel portion for all or part of the length of the foot plate, and thereby divide the toe portion into separate left and right toe parts 26A and 26B.

The foot plate is formed of a composite material which may be continuous carbon fibers in an epoxy matrix. It has resilient, spring like qualities which permit it to deform during dorsi flexion and to resiliently rebound during toe-off to transfer energy to the next step. Preferably, the foot plate is formed from layers of precured composite sheets bonded together with adhesive. The foot plate may optionally have a shock absorbing pad on all or part of the distal surface. The foot plate may also optionally have a high friction material on all or part of the distal surface for use without a shoe or other foot covering.

The top adapter 30 is the interface that is used to attach the prosthetic foot to the remainder of the prosthesis, for example to a socket part or an angular alignment adaptor. It may use an industry standard four hole pattern or any other configuration for attaching the prosthetic foot to the remainder of the prosthesis via a tube clamp, a pyramid clamp, a bolt, or any other securement device. The angular alignment structure may also be built into the top adapter. Such alignments could include slide adjustment in the anterior/posterior direction; slide adjustment in the medial/lateral direction; axial rotation; dorsi flexion/plantar flexion rotation with adjustable heel height. In addition to such standard alignment components in the prosthetic industry, the top adapter could also include special or unique alignment elements without the need for additional fasteners. The top adaptor could also include elements to aid in the suspension of the prosthetic socket to the residual limb, such as a valve for suction suspension.

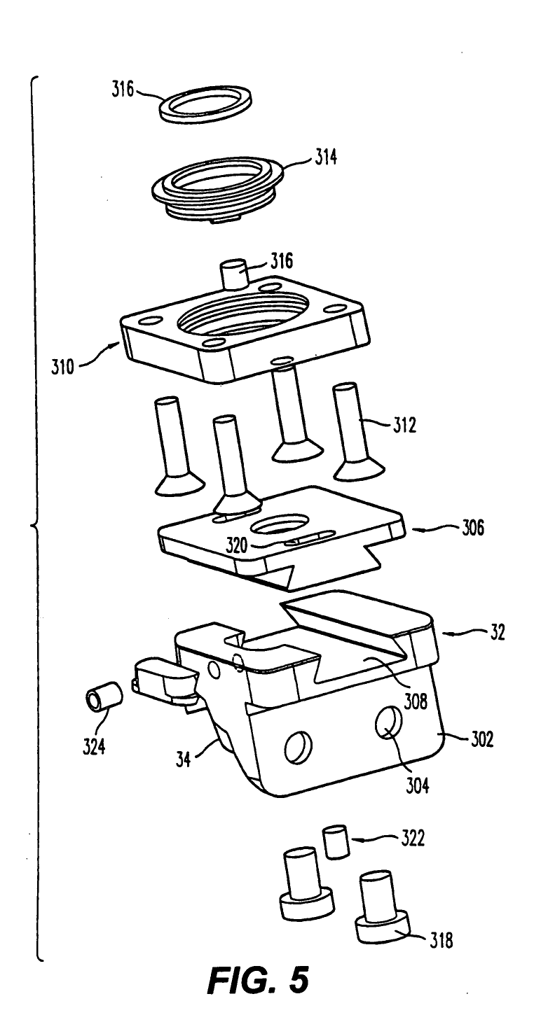

Referring particularly to FIGS. 1 and 5, the top adapter 30 preferably has a one piece cast, machined or molded top plate 32 such as a machined aluminum part including a pair of depending yokes 34 (only one is shown) via which the shock absorber 50 may be pivotally attached to the top adapter via a pair of journals 35 (only one is shown).

A downwardly directed integral shoulder 36 of the top plate 32 opposite the yokes 34 has a vertical front face 302 which provides a rear clamp support for the ends of the toe springs 40, which in the illustrated embodiment are clamped to the top adapter between the shoulder 36 and a pair of clamp plates 38. For example, screws or bolts 39 extending through the clamp plates and toe springs may be threaded into holes 304 of the shoulder 36 in order to rigidly clamp the ends of the toe springs 40 between the clamp plates 38 and the face of the shoulder 36.

A ledge 32A of the top plate extends forward of the shoulder 36 to provide an upper stop for the toe springs 40, to prevent upward movement of the compression side of the toe springs during deformation and to thereby reduce the possibility of shear failure of the toe springs in use. Also, although not illustrated in FIG. 1, the face of the shoulder 36 against which the toe springs are clamped may include serrations in order to better grip the toe springs. The clamp plates 38 may also have serrations which mate with the ribs (discussed below) of the toe springs 40. These serrations improve the clamping grip on the compression and tension sides of the toe springs to limit their relative movement during use and thereby reduce the risk of shear failure.

The top plate may be mounted to a stump socket via a dovetail slide and a rotatable adapter. For example, in this embodiment (FIG. 5) a dovetail slide member 306 has a dovetail portion which slides in a dovetail groove 308 of the top plate 32. A rotatable adaptor 310 may be screwed to a stump socket (not shown) by screws 312, and may optionally incorporate a check valve 314 and filter 316 to accommodate air expelled from the stump socket when it is mounted to a stump. The rotatable adapter 310 may be secured to the dovetail slide member 306, with rotation adjustment, by screws 318 which extend through curved slots 320 in the dovetail slide member 306, and are threaded into the rotatable adaptor 310. A slide limit screw 322 can be threaded into the top plate 32 to a position to interfere with, and limit, the movement of the slide member 306. The position of the dovetail slide member 306 in the dovetail groove 308 can be locked by tightening of a slide adjustment screw 324 threaded into the top plate 32.

Two, preferably identical, leaf springs 40 are each connected or clamped between the top adapter 30 and the toe of the foot plate 20. The clamping of one end of the toe springs to the top adapter has already been described. The opposite ends of the toe springs are similarly respectively clamped to the toe parts 26A and 26B of the foot plate 20. For example, a pair of toe spring clamps 42, which may be formed of machined aluminum, may each be bolted or riveted to a respective toe part 26A or 26B of the toe 26 of the foot plate, and may include clamp plates similar to clamp plates 38 for clamping the ends of the toe springs. To this end, the toe parts 26A and 26B may each include a hole 29 for the bolt or rivet of the clamp elements. Toe spring clamps 42 can provide or rigid or pivotal connection between toe springs 40 and toe part 26A or 26B of the toe 26 of the foot plate.

The toe spring clamps 42 are preferably shaped to form reliefs 46 at their posterior portions in order to allow the foot plate 20 to deflect around the toe spring clamps during heel strike. Optionally, they may also form anterior reliefs to allow the foot plate 20 to deflect around the toe spring clamps during toe-off. The toe spring clamps 42 maintain a spacing between the toe ends of the toe springs 40 and the foot plate so as to maximize the length of the toe springs available to deform during use. This, together with the clamping of the toe springs only at their ends, helps distribute the bending stresses along the entire length of the toe springs and reduces the possibility of failure.

The toe springs 40 themselves are each a leaf spring which is generally curved along its length to preferably have an unstressed arcuate end-to-end angle of 100 and 110, with an overall spring radius of 3 to 3.5 inches. The toe springs 40 are each preferably formed by a laminate of two or more different composite materials. Each layer can preferably be formed of any combination of carbon/epoxy; glass/epoxy; glass/vinyl ester; or carbon/vinyl ester. Preferably, the tension (convex) side of the toe spring is formed of a layer using long fibers while the compression (concave) side of the toe spring is formed of a layer using short glass or other fibers. Also preferably, the toe springs are made of layers of precured composite sheets bonded together with adhesive. The toe springs could also be made from spring metal.

As an alternative the two toe springs may be replaced by a single toe spring which is split along its length from the toe end to near the end clamped by the top adaptor 30.

According to a feature of the invention, the surface of each of the toe springs 40 on the concave side exhibits a plurality of transverse ribs 44 for distributing bending stresses along the length of the toe springs. The toe springs normally have a tendency to bend disproportionately at a region near their longitudinal mid-portions. This results in high localized shear stresses which can produce delaminization of the layers of the toe spring. Addressing this problem by simply making the toe springs thicker is not a satisfactory solution because the toe springs will then be too stiff to provide the desired degree of compliance during ambulation.

The ribs 44 instead address this problem by locally altering the compliance of the ribs along their length. For example, larger radius ribs make the springs stiffer while a smaller radius provides less stiffness. Therefore by controlling, for example, the rib radiuses and spacing along the lengths of the springs, one can distribute the deformation of the spring during ambulation over the entire length of the spring, and thereby permit a greater overall deflection of the spring while minimizing localized shear stresses at any given portion along the spring length. A typical rib radius may be 0.125 inches.

The shock absorber 50 is preferably a tube type shock absorber incorporating an air or hydraulic spring. For example, the “FLOAT” model bicycle shock absorber manufactured by Fox Racing Shox could be used as a shock absorber 50. The spring constant of this shock absorber can be adjusted by the user. One end of the shock absorber 50 is pivotally connected to the top adapter 30 via the journals 35 for rotation about an axis transverse to the length of the foot plate 20 and the length of the shock absorber. The other end of the shock absorber is pivotally connected to the foot plate 20 via a heel connector 52, for rotation about an axis transverse to the length of the foot plate 20 and the length of the shock absorber. To this end, the heel connector 52 also has yokes 52A and journals 52B. Alternatively, rotation in other planes could be achieved with the use of spherical bearings. The pivotal mounting of the shock absorber facilitates ankle rotation during walking, relieves stresses due to bending moments and minimizes the risk of binding of the shock absorber.

The heel connector can also include a posterior relief 52C to allow the foot plate to deflect around the heel connector during heel strike.

During ambulation, the shock absorber is compressed to absorb the shock of the initial heel strike for each step. The user can adjust the spring constant of the shock absorber to that required for the particular use. The pivotal mounting of the shock absorber minimizes turning moments and binding of the shock absorber. Also, since the prosthetic foot is mounted to the remainder of the prosthetic limb and the user's leg via the top adaptor 30, and not entirely via the shock absorber, any shortening of the prosthesis due to compression of the shock absorber at this time is minimized.

As the foot rotates from the heel strike towards toe-off during each step, the load is gradually transferred from the heel toward the toe of the foot plate 20, causing the foot plate to deform. This deformation of the foot plate 20 is resisted by the controlled bending deformation of the toe springs. A simultaneous elastic return of the shock absorber 50 also propels the user forward and transfers energy to the toe springs 40, causing them to further deform. The ribs 44 distribute the bending load along the length of the toe springs 40 and thereby minimize the risk of delamination or other spring failure. The shear stresses are also controlled by the tight grip of the toe springs provided by the serrations on the shoulder 25 and the clamp plates 38. The stop 32A also limits the differential movement of the compression sides of the toe springs, relative to the tension sides during bending, which also helps limit shear stresses.

The separate mounting of the toe springs 40 to the toe parts 26A and 26B permits the toe springs to individually respond to foot inversion or eversion.

Finally, during toe-off, the foot plate 20 and toe springs 40 rebound to provide energy transfer for the next step. Since the shock absorber 50 has fully returned by this time, a maximum energy transfer is possible.

The prosthetic foot of this design has several advantages over conventional prosthetic feet. The bonded layers of precured composite sheet construction of the toe spring, foot plate, and heel leaf spring increase durability by distributing stresses through the thickness of these elements. The ribbed construction of the toe springs increase their durability by distributing the shear stresses along the length of the toe springs. Since the shock absorber is in parallel with the toe spring and is pivotally connected, the shock absorber is not compressed at toe-off and so there is no damping of the toe spring energy return by the shock absorber. The generally triangular shape of the prosthesis provides a compact design, minimizes the limb shortening phenomenon and provides a smoother transition from heel to toe. The flexibility of the foot also facilitates “foot flat,” i.e., broad surface area contact of the foot with the ground, which promotes stability. The generally triangular shape of the prosthesis can also increase durability by inherently restricting the maximum deflection of the flexible components. The air spring shock absorber provides a more dynamic response than currently available shock absorbers.

In the variant of FIG. 2, the prosthetic foot 10A is identical to that of FIG. 1, except as set forth below. In particular, the shock absorber 50 is replaced by a composite leaf spring 150 which is pivotally connected to both the foot plate 20 and the top adapter 130 by journals 152 and 154. The leaf spring 150 may also be ribbed in the same way as the toe springs 40. The leaf spring may be made of layers of precured composite sheets bonded together with adhesive. The leaf spring may be exchanged for another of different spring constant for different activities. Alternatively, composite leaf spring 150 could be replaced by any spring material such as metal or polymer, including urethane.

The variant of FIG. 2 may also use a modified top adapter 130 which incorporates a toe spring angle compensation mechanism, which is useful for compensating for different shoe heel heights. To this end, the part forming the shoulder 136 is not integral with the top plate 132. Rather, the top plate 132 has a concave arcuate serrated lower surface 133 to which a serrated top end of the shoulder part 136 may be clamped by a nut or other means. By selecting the point of clamping of the shoulder part 136 along the arcuate surface 133, one can adjust the attachment angle of the toe springs to the top adapter.

While the toe spring angle compensation mechanism is shown with respect to the variant of FIG. 2, it is equally applicable to that of FIG. 1. However, the design in FIG. 1 can also compensate for heel height adjustment (e.g., for different footwear) by adjusting fluid pressure in the shock absorber.

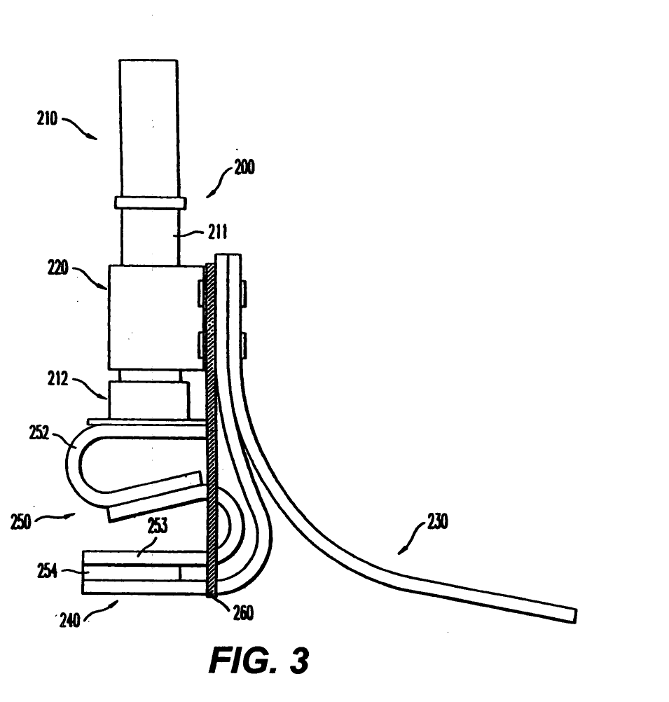

The main components of the prosthetic foot 200 according to the alternate embodiment of FIG. 3 include a pylon 210, a collar 220 slidably mounted to the pylon, a toe spring 230 forming a toe of the prosthetic foot, a heel spring 240 forming a heel of the prosthetic foot and a further, preferably S-shaped, heel spring 250 connected between the pylon 210 and the heel portion of the heel spring 230. Additionally, a band 260 made of a non-extensible material is wound around the heel spring and the collar 220 in order to limit the upward movement of the collar during the rebound of the further heel spring 250.

Turning more particularly to the specific embodiment illustrated in the FIGUREs, the pylon 210 may be a standard 30 mm tube having a top end connected to a socket in a conventional fashion. The collar 220 defines a bore (not illustrated) which closely surrounds the pylon such that the collar 220 can slide along the length of the pylon 210 without excessive friction or binding. To this end, the collar 220 is preferably made of machined aluminum, and either or both the surface of the pylon or the interior of the bore, in the area where the collar 220 reciprocates, can include a lining of low friction material as a linear bearing. For example, the pylon 210 may have a low friction material sleeve or sheathing 211.

The toe spring 230 and the heel spring 240 are fixed to the collar 220, for example by rivets or bolts. Bolts have the advantage that they permit adjustment of the position of fixation of the toe and heel springs onto the collar 220.

The toe spring 230 is preferably a composite leaf spring made from, e.g., glass or carbon fibers in an epoxy matrix. It is generally arcuate and forms the toe portion of the prosthetic foot. The heel spring 240 may be formed of the same material as the toe spring 230 and forms a heel portion of the prosthetic foot.

The further heel spring 250 is preferably S-shaped and may be formed from two C-shaped springs 252 and 253 which are bonded to one another, each of which may also be a composite spring of the same material as the toe spring 230. One end of the further spring 250 may be secured to lower end of the pylon 210, e.g., via tube clamp 212, while the other end of the further spring 250 may be fixed to the heel portion of the heel spring 240, for example by adhesively attaching the lower C-shaped spring 253 to a rubber bumper 254 which is itself adhered to the heel spring 240.

The non-extensible band 260 may be formed of Kevlar or of Spectra (a high molecular weight polyethylene manufactured by Allied Signal) loops.

During ambulation, the further heel spring 250 compresses during heel strike in order to absorb energy. When this occurs, the collar 220 is carried by the heel spring 240 and slides upwardly on the pylon 210. As foot rotation continues, the load is gradually transferred onto the toe spring 230 which is deformed thereby, and the further heel spring 250 simultaneously rebounds and transfers energy to the toe spring 230. As this occurs, the resulting load would normally cause the collar 230 to move to the top of the pylon 210, but this is prevented by the band 260.

The use of both the heel spring 240 and the further heel spring 250 provides a more natural feel and minimizes binding of the collar as it slides along the pylon.

The S-shape of the spring 250 prevents an angular change between the heel portion of the prosthetic foot and the pylon as the further heel spring 250 is compressed.

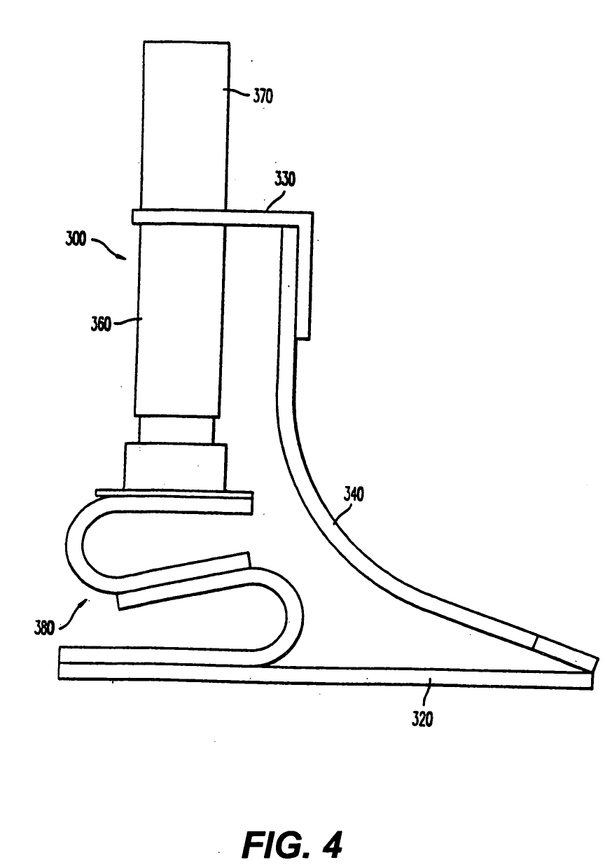

The prosthetic foot 300 according to the further embodiment schematically illustrated in FIG. 4 represents a hybrid of the first two embodiments. In this embodiment, the foot plate 320 may be identical to that shown in FIG. 1 (although, for ease of illustration, its contouring is not shown). Similarly, the toe springs 340 may be identical to those of the first embodiment, and may be clamped in the same way (although this is also not shown).

According to this embodiment, the top adapter 330 incorporates a collar 360 similar to that of the second embodiment, and which slides along a pylon 370. A heel spring 380 may be S-shaped and correspond to the further heel spring of the previous embodiment. As in the previous embodiment, a non-extensible band (not shown) limits the upward movement of the collar 360.

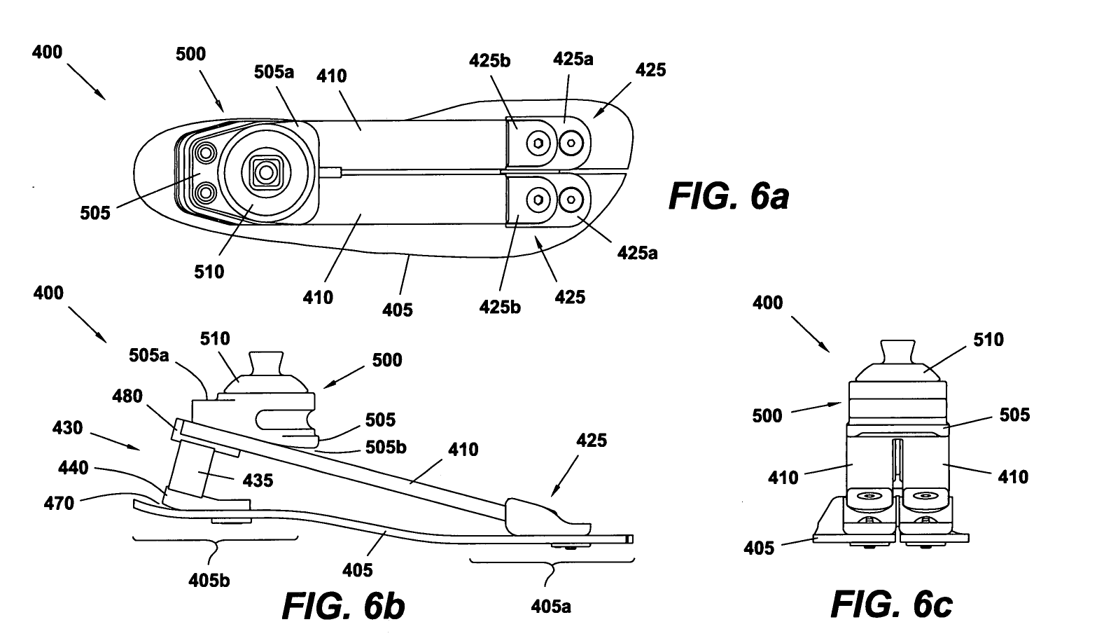

An alternate embodiment of a prosthetic foot 400 of the present invention can be observed in FIGS. 6-8. This embodiment of the prosthetic foot 400 is designed for use with a prosthetic ankle. Consequently, in order to allow for installation of the prosthetic ankle between the prosthetic foot 400 and a connecting portion of a prosthetic leg, this prosthetic foot has a lower profile than known prosthetic foot devices as well as the previously described exemplary prosthetic foot embodiments. Preferably, the prosthetic foot 400 is designed so that upon attachment of a prosthetic ankle thereto, the prosthetic ankle will be at a height (position) that approximates the height (position) of a typical human ankle.

The prosthetic foot 400 is shown to include a contoured foot plate 405, which may or may not be the same as the contoured foot plate 20 illustrated in FIGS. 1-2. In the particular embodiment of the prosthetic foot 400 depicted in FIGS. 6-8, the foot plate 405 is the same as the foot plate 20 shown in FIGS. 1-2. The prosthetic foot 400 also includes a pair of toe springs 410 and a pair of heel-mounted shock absorbing assemblies 430. The toe springs 410 may be formed by a laminate of two or more different composite materials or from a spring metal, as previously described. The pair of toe springs 410 may also be replaced by a single toe spring. When a single toe spring is employed, it may optionally be split along its length from a distal end 415 end to near a proximal end 420.

The distal end 415 of each toe spring 410 is connected near a toe portion 405a of the foot plate 405 by a toe spring clamp assembly 425. The particular embodiment of the toe spring clamp assembly 425 shown in FIGS. 6-8 includes a toe spring cup 425a and a toe spring clamp plate 425b. The toe spring cup 425a may be attached to the foot plate by a variety of means, including the threaded fastener 422, T-nut 423 and dowel rod 424 combination shown. Similarly, the toe spring clamp 425b may be caused to clamp the toe spring 410 to the toe spring cup 425a by a variety of means, including the threaded fastener 426 and T-nut 427 assembly shown. There may be a single toe spring clamp assembly 425 that is adapted to receive the distal end 415 of both toe springs 410 or, alternatively, there may be a separate toe spring clamp assembly for receiving the distal end of a respective one of the toe springs. Like the prosthetic foot 10 shown in FIGS. 1-2, the toe spring clamp assembly 425 of this embodiment may be formed of machined aluminum. The toe spring clamp assembly 425 may also be formed of another metal, or a plastic or composite material. The toe spring clamp assemblies 425 may each be bolted or riveted to the foot plate 405, and may provide for a rigid or pivotal connection between the toe springs 410 and the foot plate. The toe spring clamp assembly 425 may be of other designs as well. For example, a molded toe spring clamp assembly, as described in more detail below, may replace the toe spring clamp assembly 425 shown.

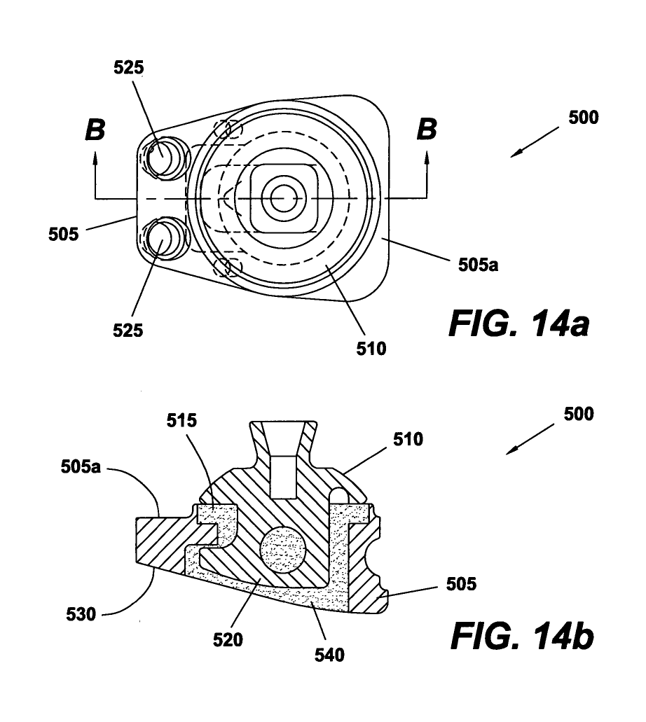

A proximal end 420 of each toe spring 410 is trapped between a prosthetic foot connection component 505 of a prosthetic ankle 500 (see FIGS. 14a and 14b) and a connecting portion of a corresponding shock absorbing assembly 430. When so mounted to the foot plate 405, the toe springs 410 will preferably extend downward at some angle from the proximal end 420 to the distal end 415 thereof.

As illustrated in FIGS. 6-8, each shock absorbing assembly 430 is comprised of a short heel spring 435, a heel attachment plate 440, and a toe spring support plate 480. These components and their associated fasteners operate to connect the proximal end 420 of the toe springs 410 to a heel portion 405b of the foot plate 405, while simultaneously contributing to the absorption of shocks developed during ambulation. When a pair of toe springs 410 is used with the prosthetic foot 400, a pair of shock absorbing assemblies 430 is also typically provided—one shock absorbing assembly per toe spring. Alternatively, a single shock absorbing assembly 430 can be used with a prosthetic foot 400 having a pair of toe springs 410. If a single toe spring is used with the prosthetic foot 400, a pair of shock absorbing assemblies 430 or a single shock absorbing assembly may be employed.

The heel spring 435 used with this embodiment of the prosthetic foot 400 may consist of, for example, a mechanical spring, or a pneumatic or hydraulic shock absorber. In the particular embodiment of the prosthetic foot 400 shown in FIGS. 6-8, the heel spring consists of a compressible elastomeric element. Such an elastomeric heel spring 435 may be of assorted cross-sectional shape. The elastomeric material used to produce such a heel spring 435 may be of various hardness. In one exemplary embodiment, the elastomeric material has a hardness value of between about 60-80 Shore A, although, depending on the particular application, the elastomeric material could also have a hardness that falls outside of this range.

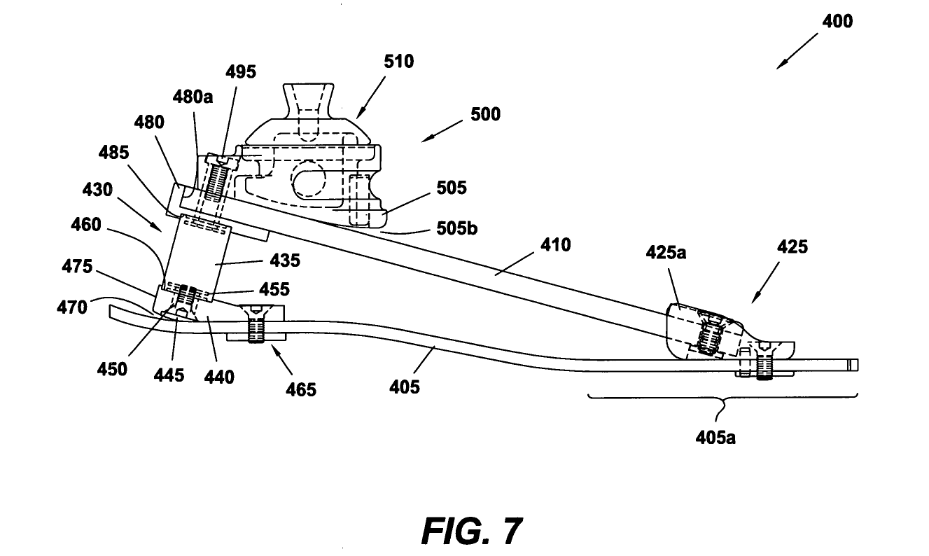

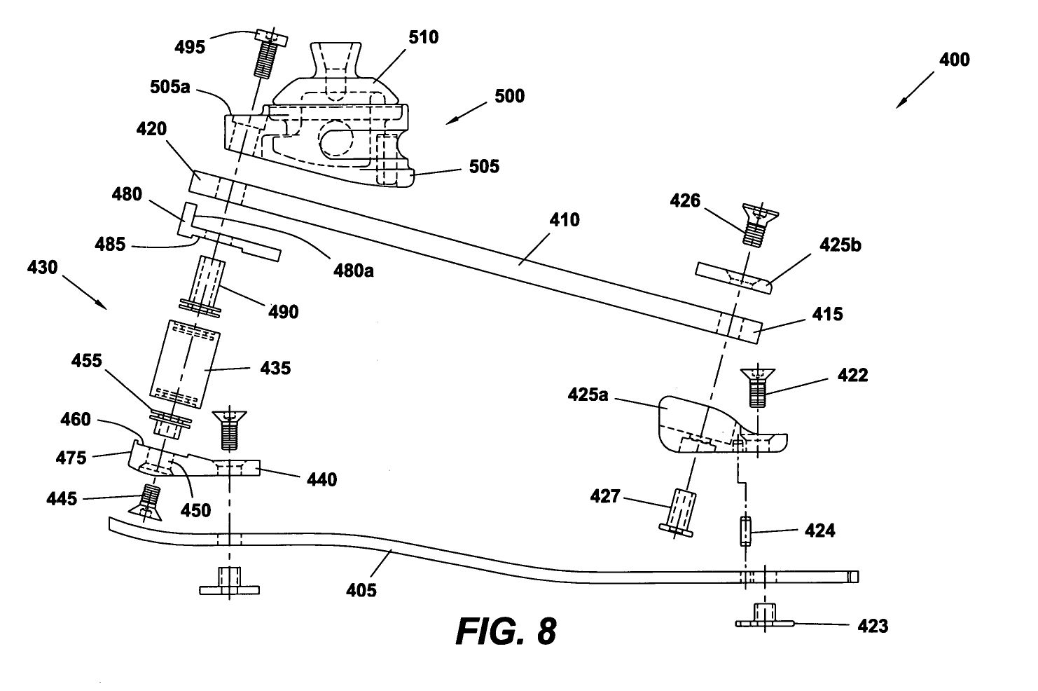

The heel attachment plate 440 facilitates affixation of one end of the heel spring 435 to the foot plate 405. The heel spring 435 may be affixed to the heel attachment plate 440 by passing a threaded fastener 445 through a recessed opening 450 in the heel attachment plate and into a correspondingly threaded hole in the heel spring. When using an elastomeric heel spring 435, as shown, a T-nut 455 may be inserted or otherwise embedded in the elastomeric material to satisfactorily receive and retain the like-threaded fastener 445. A recess 460 may be provided in the heel attachment plate 440 to receive a portion of the heel spring 435, or the heel spring may simply abut the heel attachment plate. The heel attachment plate 440 can be subsequently attached to the foot plate 405 by riveting, or by using a threaded fastener assembly 465, such as the screw and T-nut shown in FIGS. 7 and 8. The heel attachment plate 440 may also be shaped to form a relief 470 along its rearward portion 475, thereby allowing the foot plate 420 to deflect smoothly around the heel attachment plate during heel strike.

Although it may be possible to affix the heel spring 435 directly to the toe spring 410, a toe spring support plate 480 preferably resides between the other end of the heel spring and the underside of the toe spring. The toe spring support plate 480 (also referred to as a mid-plate) may have a recess 485 for receiving a portion of the heel spring 435, or the heel spring may simply abut the toe spring support plate. The heel spring 435 and the toe spring support plate 480 are preferably affixed to the toe spring 410 via a fastener extending downward from the prosthetic ankle 500. For example, in the particular embodiment of the prosthetic foot 400 shown in FIGS. 6-8, one end of a T-nut 490 is embedded in the elastomeric heel spring 435. An upwardly extending threaded section of the T-nut 490 passes through an aperture in both the toe spring support plate 480 and toe spring 410, and enters into a bore 525 in the prosthetic ankle 500. A threaded fastener 495 can then be used to draw the heel spring 435, toe spring support plate 480, toe spring 410, and prosthetic ankle 500 tightly together.

The toe spring support plate 480 may include a stop 480a that limits the differential movement of the side of the toe spring subjected to compression during bending thereof. It is believed that limiting movement of the toe spring 410 in this manner helps to limit shear stresses produced in the toe spring.

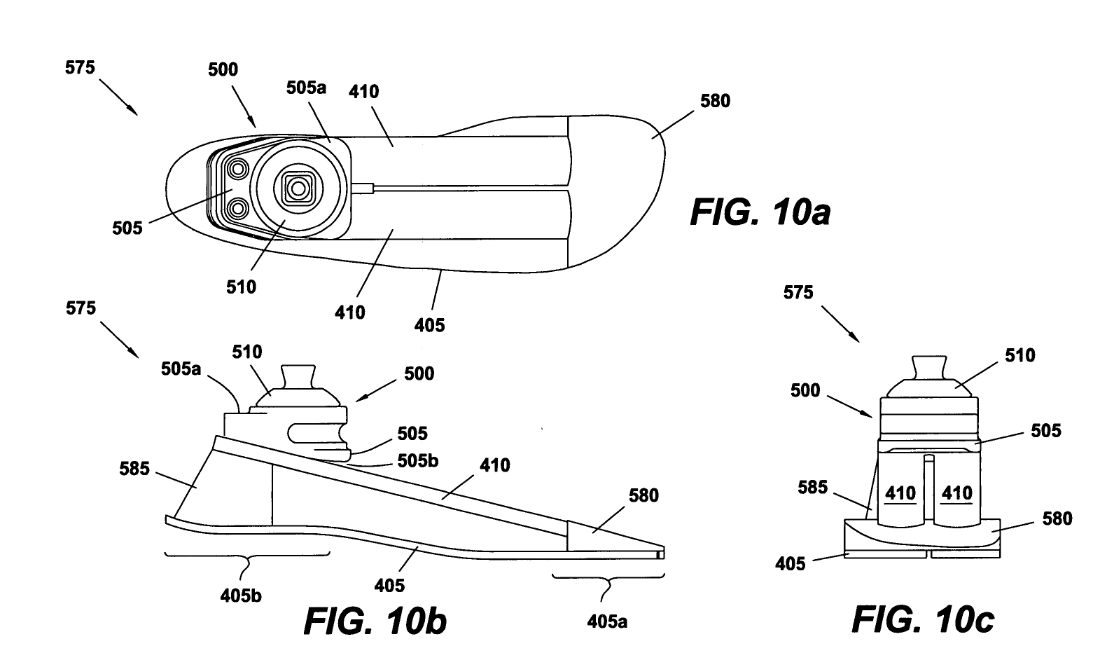

It is contemplated that various prosthetic ankles may be used with the prosthetic foot 400 of the present invention. One particular prosthetic ankle 500 that is especially well-suited for use with the prosthetic foot 400 of the present invention is illustrated in FIGS. 10-20 of U.S. application Ser. No. 10/770,883, filed on Feb. 3, 2004, and entitled Multi-Axis Prosthetic Ankle Joint. U.S. application Ser. No. 10/770,883 is herein incorporated by reference in its entirety. This multi-axis prosthetic ankle 500 is shown in FIGS. 14a-14b and can be seen to include a bottom, prosthetic foot connection component 505, that is adapted for attachment to a prosthetic foot, such as the prosthetic foot 400 shown in FIGS. 6-8. The multi-axis prosthetic ankle 500 is also shown to include a lower leg connection component 510 that is adapted to attach the ankle to the socket component of a prosthetic leg.

The prosthetic foot connection component 505 of the multi-axis ankle 500 is essentially a box-like structure having rigid vertical walls that bound a receiving cavity. The prosthetic foot connection component 505 may also form a relief 505b at its anterior portion to allow for unimpeded deflection of the toe spring(s) 410 during toe-off. The receiving cavity 515 of the prosthetic foot connection component 505 is designed to receive a connecting projection 520 of the lower leg connection component 510. More specifically, the receiving cavity 515 of the prosthetic foot connection component 505 is designed to allow the connecting projection 520 of the lower leg connection component 510 to floatingly reside therein when the two components are properly assembled. This design allows the positional relationship between the prosthetic foot connection component 505 and the lower leg connection component 510 to be maintained by an elastomeric material 540 placed in the receiving cavity, as opposed to a direct mechanical connection between the two components. During ambulation of the user, the elastomeric material 540 allows for limited movement of the connecting projection 520 within the receiving cavity 515, thereby providing for controlled multi-axis flexion of the prosthetic ankle 500.

The bore(s) 525 provided through the prosthetic foot connection component 505 of the multi-axis ankle 500 of FIGS. 16a-16b facilitate its attachment to the prosthetic foot 400, as described above. A bottom surface 530 of the prosthetic foot connection component 505 is also preferably angled to allow for proper orientation of the multi-axis prosthetic ankle 500 to the angled toe springs 410 of the prosthetic foot 400. More specifically, the angled bottom surface 530 of the prosthetic foot connection component 505 preferably allows the prosthetic ankle 500 to be mounted to the angled toe springs 410 while simultaneously maintaining a top surface 505a of the prosthetic foot connection component 505 in a substantially level position. Thus, subsequent connection of the prosthetic ankle 500 to a receiving portion of a prosthetic leg is facilitated.

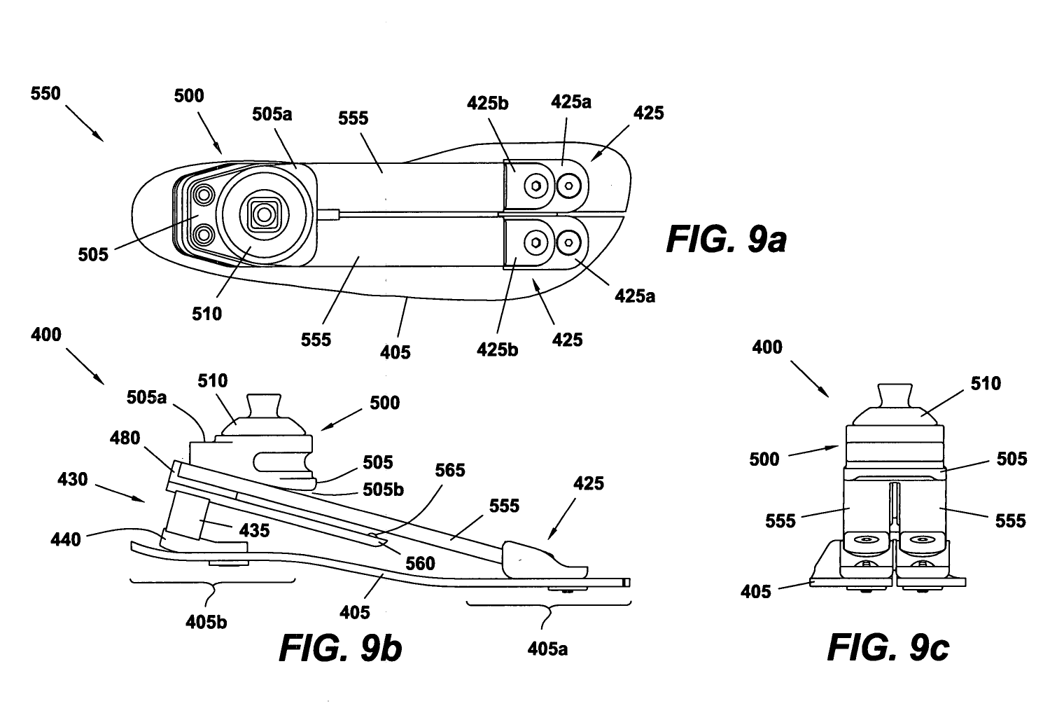

An alternate embodiment 550 of a prosthetic foot of the present invention is illustrated in FIGS. 9a-9c. Embodiments of this prosthetic foot 550 are substantially the same as embodiments of the prosthetic foot 400 shown in FIGS. 6-8, the exception being the addition of one or more auxiliary toe springs 560. When the prosthetic foot 550 utilizes a pair of (primary) toe springs 555, an auxiliary toe spring 560 is preferably provided to correspond to each primary toe spring. When the prosthetic foot 550 utilizes a single primary toe spring 555, one, or a pair, of auxiliary toe springs 560 may be provided. The primary toe springs 555 of this embodiment may be the same as the (primary) toe springs 410 of FIGS. 6-8.

The auxiliary toe spring 560 may be constructed in a similar manner to the toe springs 410 described previously. The spring force produced by the auxiliary toe spring 560 may be similar to, or different than, the spring force provided by the primary toe spring 555 of the prosthetic foot 550. The purpose of the auxiliary toe spring 560 is to help resist bending of the prosthetic foot 550 by inhibiting bending of the primary toe spring 555 once the bending thereof reaches a predetermined amount. A bumper 565 may be located between the primary and auxiliary toe springs 555, 560 to prevent direct contact therebetween. The bumper 565 is preferably constructed from an elastomeric material, but could also be another type of spring.

Another embodiment of a prosthetic foot 575 of the present invention can be observed in FIGS. 10a-10c. Embodiments of this prosthetic foot 575 are substantially similar to the embodiments of the prosthetic foot 400 shown in FIGS. 6-8, the exception being that the multi-component shock absorbing assemblies 430 and toe spring clamp assemblies 425 thereof have been replaced by one-piece molded structures 580, 585, respectively.

The prosthetic foot 575 may employ the same contoured foot plate 405 and toe springs 410 used by the prosthetic foot 400 of FIGS. 6-8. A proximal end 420 of each toe spring 410 is again trapped between a prosthetic foot connection component 505 of a prosthetic ankle 500 (see FIGS. 14a and 14b) and a connecting portion of a corresponding shock absorbing assembly 585. When so mounted to the foot plate 405, the toe springs 410 will again preferably extend downward at some angle from the proximal end 420 to the distal end 415 thereof. Similarly, the pair of toe springs 410 may again be replaced by a single toe spring, which may optionally be split along its length from a distal end 415 end to near a proximal end 420.

In this embodiment, the distal end 415 of each toe spring 410 is connected near a toe portion 405a of the foot plate 405 by a unitary toe spring clamp assembly 580—although a separate such assembly could also be provided for each toe spring. The unitary toe spring clamp assembly 580 is preferably molded to the prosthetic foot 575 with the foot plate 405 and toe springs 410 held in proper orientation to each other—such as in a specialized mold or die. In order to increase bonding therebetween, holes, cavities, or other such features (not shown) may be provided on the portion of the toe springs 410 that are encased by the material of the unitary toe spring clamp assembly 580. Texturing, knurling, or other techniques may be similarly applied to the toe springs 410 to increase adhesion and bonding between the toe springs and the unitary toe spring clamp assembly 580. Holes, cavities, or other such features (not shown) are also preferably provided through or in the foot plate 405 to increase bonding between the foot plate and the unitary toe spring clamp assembly 580. Preferably, the unitary toe spring clamp assembly 580 is molded from a flexible material, such as an elastomer. The material may have a hardness within the range previously described.

As illustrated in FIGS. 10a-10c, the multiple component shock absorbing assembly 430 of the previously described prosthetic foot 400 has been replaced by a unitary shock absorber 585. The unitary shock absorber 585 is preferably molded to the prosthetic foot 575 with the foot plate 405 and toe springs 410 held in proper orientation to each other—such as in the same mold or die used to mold the unitary toe spring clamp assembly 580 and, preferably, concurrently therewith. In order to increase bonding therebetween, holes, cavities, or other such features (not shown) may be provided on the portion of the toe springs 410 that are in contact with the material of the unitary shock absorber 585. Texturing, knurling, or other techniques may be similarly applied to the toe springs 410 to increase adhesion and bonding between the toe springs and the unitary shock absorber 585. Holes, cavities, or other such features (not shown) are also preferably provided through or in the foot plate 405 to increase bonding between the foot plate and the unitary shock absorber 585. Preferably, the unitary shock absorber 585 is molded from an elastomeric material. The elastomeric material may have a hardness within the range previously described, and may be the same as the material used to mold the unitary toe spring clamp assembly 580. The unitary shock absorber 585 may be of cross-sectional shapes other than that shown.

In a manner similar to the prosthetic foot 550 shown in FIGS. 9a-9c, an auxiliary toe spring (not shown) may be installed to the prosthetic foot 575. One, or a pair of auxiliary toe springs may be provided. The auxiliary toe spring(s) are again preferably disposed below the primary toe spring(s) 410, and are connected at one end to the unitary shock absorber 585. For example, one end of the auxiliary toe spring(s) may be molded into the unitary shock absorber 585. A spacer plate may be provided between the primary toe spring(s) 410 and the auxiliary toe spring(s). The spacer plate may be attached to the toe springs 410 and/or the unitary shock absorber 585.

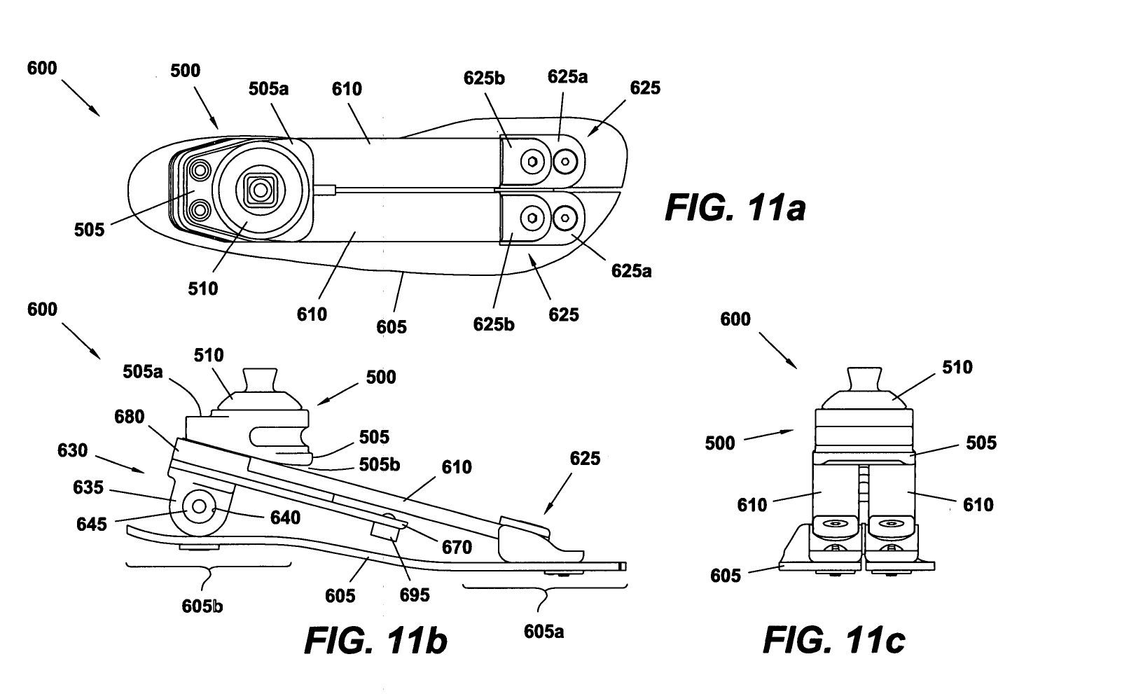

Yet another embodiment of a prosthetic foot 600 according to the present invention can be seen by reference to FIGS. 11-13. Like the prosthetic foot 400 depicted in FIGS. 6-10, this embodiment of the prosthetic foot 600 is designed for use with a prosthetic ankle. Consequently, in order to allow for installation of the prosthetic ankle between the prosthetic foot 600 and a connecting portion of a prosthetic leg, this prosthetic foot has a lower profile than the previously described exemplary prosthetic foot embodiments. Preferably, the prosthetic foot 600 is designed so that upon attachment of a prosthetic ankle thereto, the prosthetic ankle will be at a height (position) that approximates the height (position) of a typical human ankle.

Embodiments of this prosthetic foot include a contoured foot plate 605, which may or may not be the same as the contoured foot plate 405 illustrated in FIGS. 6-8. In the particular embodiment of the prosthetic foot 600 depicted in FIGS. 11-13, the foot plate 605 is the same as the foot plate 405 shown in FIGS. 6-8. In a similar manner to the prosthetic foot 400 of FIGS. 6-8, this embodiment of the prosthetic foot 600 also includes a pair of toe springs 610 and a heel-mounted shock absorber 630. The toe springs 610 may be formed as previously described, and may also be replaced by a single toe spring that may optionally be split along its length from a distal end 615 to near a proximal end 620. The distal end 615 of each toe spring 610 is again preferably connected near a toe portion 605a of the foot plate 605 by a toe spring clamp assembly 625. The particular embodiment of the toe spring clamp assembly 625 shown in FIGS. 11-13 may include a toe spring cup 625a and a toe spring clamp plate 625b. The toe spring cup 625a may be attached to the foot plate by a variety of means, including the threaded fastener 622, T-nut 623 and dowel rod 624 combination shown. Similarly, the toe spring clamp 625b may be caused to clamp the toe spring 610 to the toe spring cup 625a by a variety of means, including the threaded fastener 626 and T-nut 627 assembly shown. The toe spring clamp assembly 625 may be of other designs as well, such as, without limitation, a unitary design similar to or the same as that described above with reference to the prosthetic foot 575 shown in FIGS. 10a-10c.

A proximal end 620 of each toe spring 610 is trapped between a prosthetic foot connection component of a prosthetic ankle (see the ankle 500 of FIGS. 14a and 14b, for example) and a connecting portion of a corresponding shock absorber 630. When so mounted to the foot plate 605, the toe springs 610 of this prosthetic foot 600 will also preferably extend downward at some angle from the proximal end 620 to the distal end 615 thereof.

This particular embodiment of the prosthetic foot 600 is shown to make use of one or more optional auxiliary toe springs 670, although it should also be realized that the auxiliary toe spring(s) could be omitted and the shock absorber 630 attached directly to the toe springs 610. In this embodiment, each auxiliary toe spring 670 is disposed beneath the (primary) toe spring(s) 610, and acts to produce increased resistance to bending when overall bending of the prosthetic foot 600 reaches a predetermined point. The auxiliary toe spring 670 may be separated from the toe spring 610 by means of an optional spacer plate 680. The spacer plate 680 may extend some distance along the length of the auxiliary toe spring 670. The spacer plate 680 may have a cavity or recess 685 for receiving a portion of the toe spring 610. Alternatively, the spacer plate 680 may be substantially flat (without a cavity or recess). An optional stop 690 may be located at the anterior of the spacer plate 680 to limit the differential movement of the side of the toe spring(s) 610 subjected to compression during bending thereof. The spacer plate 680 can be made from various materials, including rigid materials and compressible materials having different compressive moduli. A stop 695 may be attached near one end of the auxiliary toe spring(s) 670 to further limit bending of the prosthetic foot 600 through contact with the foot plate 610. The stop 695 may also prevent contact between the end of the auxiliary toe spring 670 and the (primary) toe spring 610. The stop 695 may be constructed from a rigid material but, preferably, is constructed from an elastomeric material.

As illustrated in FIGS. 11-13, the shock absorber 630 of this embodiment of the prosthetic foot 600 includes a substantially U-shaped heel spring 635, the rounded end 635b of which is adapted for connection to the foot plate 605, while the opposing flat end 635a is adapted for connection to the toe springs 610 (or an intermediary element).

In the particular embodiment of the prosthetic foot 600 shown, the flat end 635a of the heel spring 635 actually abuts the pair of auxiliary toe springs 670, which lie subjacent to the spacer plate 680 that abuts the bottom side of the (primary) toe springs 610. Thus, the spacer plate 680, auxiliary toe springs 670, and heel spring 635 are trapped in a stacked relationship between the bottom surface of the toe springs 610 and the top surface of the foot plate 605, thereby (along with associated fasteners) connecting the proximal ends 620 of the toe springs to a heel portion 605b of the foot plate. The rounded end 635b of the heel spring 635 allows for unrestrained bending of the heel portion 605b of the foot plate 605. In other embodiments of a prosthetic foot, the flat end 635a of the heel spring 635 may be in direct contact with the toe springs 610.

The shock absorber 630 may employ a heel spring 635 comprising, for example, a mechanical spring, or a pneumatic or hydraulic shock absorber. In the particular embodiment of the prosthetic foot 600 shown in FIGS. 11-13, however, the heel spring 635 consists of a compressible elastomeric element. The elastomeric material may have a hardness value within the range previously described, although elastomeric materials having other hardness values can also be used. The shock absorber 630 may consist of a solid elastomeric heel spring 635 formed from a single elastomeric material. In order to more accurately control the reaction of the heel spring 635 to compression forces, however, the heel spring may also be a solid element made up of more than one elastomeric material, with each material possessing different compressive properties.

Alternatively, as shown, the shock absorbing properties of the heel spring 635 may be more precisely controlled by providing a recess(es) or aperture(s) 640 of various size therein. The heel spring 635 may be used in such a condition, or a secondary heel spring element 645 may be located in the recess or aperture 640. For example, in the embodiment of the prosthetic foot 600 shown in FIGS. 11-13, a secondary heel spring element 645 formed of a material having a preferably different compressive modulus of elasticity (compressive modulus) than the elastomeric material of the remainder of the heel spring 635 is inserted into the aperture 640. More specifically, the overall compressive (shock absorbing) properties of the shock absorber 630 can be selectively affected by employing a secondary heel spring element 645 formed of a material whose compression modulus is greater or less than the compression modulus of the elastomeric material of the remainder of the heel spring 635. It has been found that secondary heel spring element 645 materials having a hardness value of between about 10-90 Shore A may be successfully used to provide for the wide variety of overall shock absorber 630 compressive values commonly required by different amputees. Of course, secondary heel spring element 645 materials having a hardness value outside of this range may also be used, depending on the particular amputee and the material properties of the remainder of the heel spring 635. In this manner, the shock absorber 630 can employ a single primary heel spring 635 for all patients, with heel compression adjustments for individual patients accomplished through the selection and use of the secondary heel spring element 645.

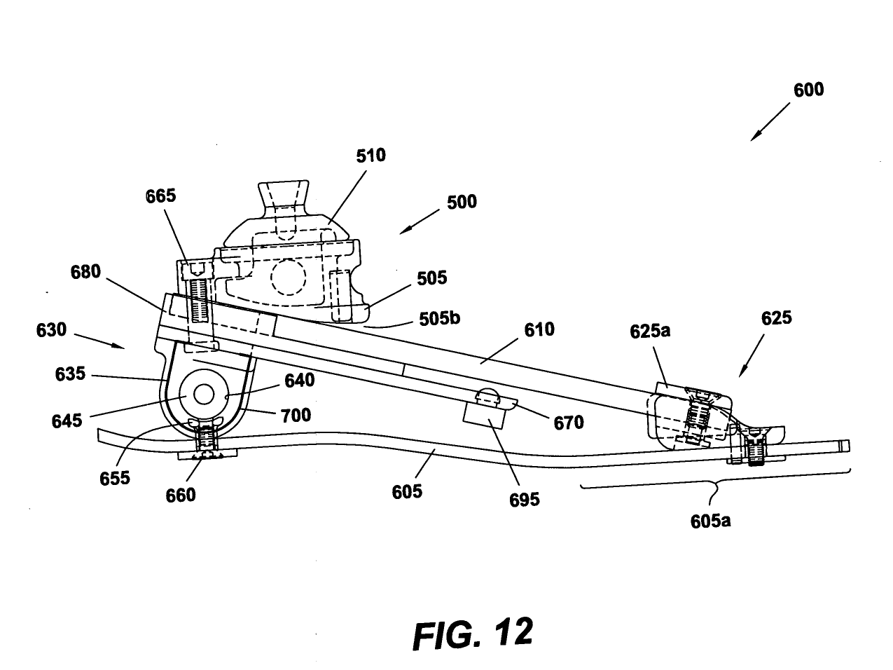

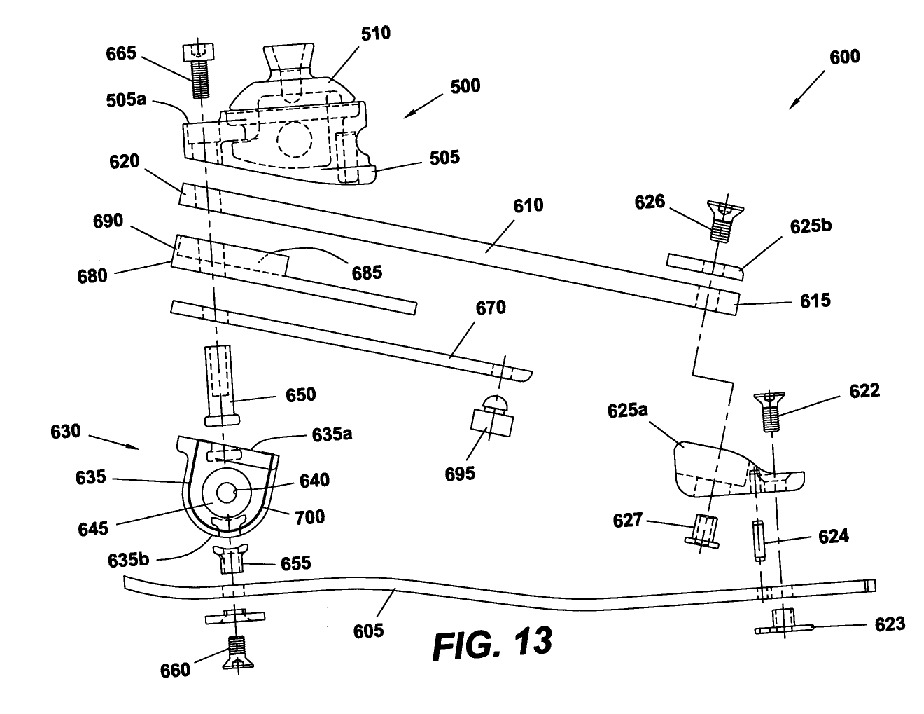

When an elastomeric heel spring 635 is used, a fiber reinforcement material 700 may be located in the elastomeric material to increase the strength of the heel spring. The fiber reinforcement material 700 prevents the elastomeric material form exceeding its elastic limit. As shown in FIGS. 12 and 13, the fiber reinforcement material 700 forms a layer of some width and thickness that preferably resides subjacent to the outer surface of the heel spring 635 and substantially follows the contour thereof. Of course, other shapes and orientations of the fiber reinforcement material 700 are also possible, and such are considered within the scope of the present invention. The fiber reinforcement material 700 may be comprised of one or more materials. While a variety of materials may be acceptably used, it has been found that Kevlar®, available from DuPont, and Spectra®, available from Honeywell, provide for good results when selected to form the fiber reinforcement layer 700.

When using an elastomeric heel spring 635, as shown, a T-nut 650, 655 or similar fastening element may be inserted or otherwise embedded in one or both ends of the elastomeric material to satisfactorily receive and retain a like-threaded fastener 660. The shock absorber 630 can be subsequently attached to the foot plate 605 by passing the threaded fastener 660 through the foot plate and into the T-nut 655. Alternatively, the shock absorber 630 can be attached to the foot plate 605 by riveting, or by various other known fastening methods. In this embodiment of the prosthetic ankle 500, the toe spring(s) 610, optional spacer plate 680, and optional auxiliary toe spring 670 may be attached to the shock absorber 630 by passing a threaded fastener 665 through bore(s) therein and into the T-nut 650. Obviously, other means of fastening the various components of the prosthetic foot 600 are also possible. For example, threaded male fasteners may be embedded in the elastomeric heel spring 635 and allowed to protrude therefrom. Thereafter, threaded female fasteners may be used to secure the various applicable components thereto.

It should be realized that the single shock absorber 630 shown in FIGS. 11-13 could also be replaced by a pair (or more) of shock absorbers. For example, an individual shock absorber may be provided for each toe spring 610 of the prosthetic foot 600.

It is contemplated that various prosthetic ankles may be used with the prosthetic foot 600 of the present invention. However, it has been found that the articulating prosthetic ankle 500 illustrated in FIGS. 14a and 14b and described in detail above is especially well-suited for use with the prosthetic foot 600 of the present invention.

The bores 525 provided through the prosthetic foot connection component 505 of the multi-axis ankle 500 facilitate its attachment to the prosthetic foot 600, as previously described. A bottom surface 530 of the prosthetic foot connection component 505 is again preferably angled to allow for proper orientation of the multi-axis prosthetic ankle 500 to the angled toe springs 610 of the prosthetic foot 600. More specifically, the angled bottom surface 530 of the prosthetic foot connection component 505 preferably allows the prosthetic ankle 500 to be mounted to the angled toe springs 610 while simultaneously maintaining a top surface 505a of the prosthetic foot connection component 505 in a substantially level position. Thus, subsequent connection of the prosthetic ankle 500 to a receiving portion of a prosthetic leg is facilitated.

While the preceding exemplary embodiments have been shown and described with respect to their use of the prosthetic ankle 500 shown in FIGS. 14a-14b, it is to be understood that other prosthetic ankle designs may also be successfully used with a prosthetic foot of the present invention. For example, more simplistic ankle designs may also be acceptably used with a prosthetic foot of the present invention. More specifically, prosthetic ankles with a single axis or limited axes of movement, or prosthetic ankles of substantially fixed position, can also be used. In any case, however, once attached to the prosthetic foot, the position (height) of the prosthetic ankle preferably approximates the position (height) of a typical human ankle.

While certain embodiments of the present invention are described in detail above, the scope of the invention is not to be considered limited by such disclosure, and modifications are possible without departing from the spirit of the invention as evidenced by the following claims:

1. A low profile prosthetic foot for use with a prosthetic ankle, comprising:
a foot plate having a heel portion and a toe portion spaced along the length thereof;
at least one toe spring having a distal end connected to said toe portion of said foot plate and a proximal end residing some distance above said heel portion of said foot plate; and
at least one heel spring having a first end connected to said heel portion of said foot plate and extending upward such that a second end thereof is connected near said proximal end of said at least one toe spring;
wherein, when connected to said prosthetic foot, the position of a prosthetic ankle will approximate the position of a typical human ankle.

2. The low profile prosthetic foot of claim 1, wherein a pair of toe springs are connected between said toe portion of said foot plate and said at least one heel spring.

3. The low profile prosthetic foot of claim 1, wherein a pair of heel springs are connected between said heel portion of said foot plate and said proximal end of said at least one toe spring.

4. The low profile prosthetic foot of claim 1, further comprising a heel attachment plate disposed between said foot plate and said at least one heel spring.

5. The low profile prosthetic foot of claim 1, further comprising a toe spring support plate disposed between said at least one heel spring and said at least one toe spring.

6. The low profile prosthetic foot of claim 5, wherein said toe spring support plate includes a stop that limits the differential movement of a side of said at least one toe spring that is subjected to compression during bending thereof.

7. The low profile prosthetic foot of claim 1, wherein said at least one heel spring is a pneumatic or hydraulic shock absorber.

8. The low profile prosthetic foot of claim 1, wherein said at least one heel spring is a helical compression spring.

9. The low profile prosthetic foot of claim 1, wherein said at least one heel spring is made from an elastomeric material.

10. The low profile prosthetic foot of claim 9, wherein said at least one heel spring has at least one recess or cavity located therein, said recess or cavity affecting the compressive resistance of said at least one heel spring.

11. The low profile prosthetic foot of claim 10, wherein a secondary heel spring element is located in said recess or cavity, said secondary heel spring element having a compressive modulus that differs from the compressive modulus of the elastomeric material forming said at least one heel spring.

12. The low profile prosthetic foot of claim 9, wherein said at least one heel spring is a U-shaped element, a rounded end of which is adapted for connection to said foot plate.

13. The low profile prosthetic foot of claim 9, further comprising a threaded fastener embedded in one, or both, ends of said at least one heel spring.

14. The low profile prosthetic foot of claim 9, further comprising a fiber reinforcement material embedded in said elastomeric material.

15. The low profile prosthetic foot of claim 1, further comprising at least one auxiliary toe spring disposed below said at least one toe spring.

16. The low profile prosthetic foot of claim 15, further comprising a spacer plate disposed between said at least one toe spring and said at least one auxiliary toe spring.

17. The low profile prosthetic foot of claim 16, further comprising a recess in said spacer plate for receiving a proximal portion of said at least one toe spring.

18. The low profile prosthetic foot of claim 17, wherein said recess acts to limit the differential movement of a side of said at least one toe spring that is subjected to compression during bending thereof.

19. The low profile prosthetic foot of claim 16, further comprising a stop on said spacer plate that limits the differential movement of a side of said at least one toe spring that is subjected to compression during bending thereof.

20. The low profile prosthetic foot of claim 1, wherein said at least one toe spring is secured to said at least one heel spring by passing at least one fastener through a prosthetic ankle, through said at least one toe spring, and into said at least one heel spring.

21. The low profile prosthetic foot of claim 1, wherein said at least one toe spring is molded to one end of said at least one heel spring.

22. The low profile prosthetic foot of claim 1, wherein one end of said at least one heel spring is molded to said foot plate.

23. The low profile prosthetic foot of claim 1, wherein said distal end of said at least one toe spring is connected to said foot plate by a toe spring clamp assembly that is molded to said foot plate.

24. A low profile prosthetic foot assembly, comprising:
a foot plate having a heel portion and a toe portion spaced along the length thereof;
at least one toe spring having a distal end connected to said toe portion of said foot plate, said at least one toe spring extending upward at an angle to a proximal end located above said heel portion of said foot plate;
at least one heel attachment plate, said at least one heel attachment plate adapted for connection to said heel portion of said foot plate and to one end of a heel spring;
at least one toe spring support plate, said at least one toe spring support plate adapted for connection to a proximal end of said at least one toe spring and to an opposite end of a heel spring;
at least one heel spring, said at least one heel spring extending upward from said foot plate toward said at least one toe spring and disposed between said at least one heel attachment plate and said at least one toe spring support plate;
a fastener for connecting a prosthetic ankle, said proximal end of said at least one toe spring, and said at least one toe spring support plate to said at least one heel spring;
a fastener for connecting said heel portion of said foot plate and said at least one heel attachment plate to said at least one heel spring; and
a prosthetic ankle adapted for mounting to said prosthetic foot;
wherein, when connected to said prosthetic foot, the position of said prosthetic ankle will approximate the position of a typical human ankle.

25. The low profile prosthetic assembly foot of claim 24, wherein a pair of toe springs are connected between said toe portion of said foot plate and said at least one heel spring.

26. The low profile prosthetic foot assembly of claim 24, wherein a pair of heel springs are connected between said heel portion of said foot plate and said proximal end of said at least one toe spring.

27. The low profile prosthetic foot assembly of claim 24, wherein said toe spring support plate includes a stop that limits the differential movement of a side of said at least one toe spring that is subjected to compression during bending thereof.

28. The low profile prosthetic foot assembly of claim 24, wherein said at least one heel spring is a pneumatic or hydraulic shock absorber.

29. The low profile prosthetic foot assembly of claim 24, wherein said at least one heel spring is a helical compression spring.

30. The low profile prosthetic foot assembly of claim 24, wherein said at least one heel spring is made from an elastomeric material.

31. The low profile prosthetic foot assembly of claim 30, wherein said at least one heel spring has at least one recess or cavity located therein, said recess or cavity affecting the compressive resistance of said at least one heel spring.

32. The low profile prosthetic foot assembly of claim 31, wherein a secondary heel spring element is located in said recess or cavity, said secondary heel spring element having a compressive modulus that differs from the compressive modulus of the elastomeric material forming said at least one heel spring.

33. The low profile prosthetic foot assembly of claim 30, further comprising a threaded fastener embedded in one, or both, ends of said at least one heel spring.

34. The low profile prosthetic foot assembly of claim 30, further comprising a fiber reinforcement material embedded in said elastomeric material.

35. The low profile prosthetic foot assembly of claim 30, further comprising at least one auxiliary toe spring disposed below said at least one toe spring.

36. The low profile prosthetic foot assembly of claim 35, further comprising a spacer plate disposed between said at least one toe spring and said at least one auxiliary toe spring.

37. The low profile prosthetic foot assembly of claim 36, further comprising a recess in said spacer plate for receiving a proximal portion of said at least one toe spring.

38. The low profile prosthetic foot assembly of claim 37, wherein said recess acts to limit the differential movement of a side of said at least one toe spring that is subjected to compression during bending thereof.

39. The low profile prosthetic foot assembly of claim 36, further comprising a stop on said spacer plate that limits the differential movement of a side of said at least one toe spring that is subjected to compression during bending thereof.

40. The low profile prosthetic foot assembly of claim 24, wherein said at least one toe spring is secured to said at least one heel spring by passing at least one fastener through a prosthetic ankle, through said at least one toe spring, and into said at least one heel spring.

41. The low profile prosthetic foot assembly of claim 24, wherein said distal end of said at least one toe spring is connected to said foot plate by a toe spring clamp assembly that is molded to said foot plate.

42. A low profile prosthetic foot assembly, comprising:
a foot plate having a heel portion and a toe portion spaced along the length thereof;
at least one primary toe spring having a distal end connected to said toe portion of said foot plate, said at least one toe spring extending upward at an angle to a proximal end located above said heel portion of said foot plate;
at least one auxiliary toe spring having a distal end and a proximal end and disposed beneath said at least one primary toe spring;
a spacer plate residing between proximal ends of said at least one primary toe spring and said at least one auxiliary toe spring;
at least one heel spring, said at least one heel spring constructed from an elastomeric material and having a recess or cavity for receiving a secondary heel spring element made from a material having a compressive modulus differing from that of said elastomeric material, a rounded end of said heel spring connected to said foot plate and an opposite, flat end, of said heel spring abutting said at least one auxiliary toe spring;
a fastener for connecting an articulating ankle, said proximal end of said at least one toe spring, said spacer plate, and said proximal end of said at least one auxiliary toe spring to said at least one heel spring;
a fastener for connecting said heel portion of said foot plate to said at least one heel spring; and
a prosthetic ankle adapted for mounting to said prosthetic foot;
wherein, when connected to said prosthetic foot, the position of said prosthetic ankle will approximate the position of a typical human ankle.

43. The low profile prosthetic foot assembly of claim 42, further comprising a recess in said spacer plate for receiving a proximal portion of said at least one toe spring.

44. The low profile prosthetic foot assembly of claim 43, wherein said recess acts to limit the differential movement of a side of said at least one toe spring that is subjected to compression during bending thereof.

45. The low profile prosthetic foot assembly of claim 42, further comprising a stop on said spacer plate that limits the differential movement of a side of said at least one toe spring that is subjected to compression during bending thereof.

46. The low profile prosthetic foot assembly of claim 42, further comprising a stop located near said distal end of said at least one auxiliary toe spring, said stop limiting total bending of said toe springs by contacting said foot plate.

47. The low profile prosthetic foot assembly of claim 46, wherein said stop also prevents contact between said distal end of said at least one auxiliary toe spring and said at least one primary toe spring.

48. The low profile prosthetic foot assembly of claim 42, further comprising a fiber reinforcement material embedded in said heel spring.

49. A low profile prosthetic foot assembly, comprising:
a foot plate having a heel portion and a toe portion spaced along the length thereof;
at least one toe spring having a distal end connected to said toe portion of said foot plate, said at least one toe spring extending upward at an angle to a proximal end located above said heel portion of said foot plate;
a toe spring clamp assembly molded to said toe portion of said foot plate and connecting said at least one toe spring thereto;
at least one elastomeric heel spring having one end thereof molded to at least a portion of said proximal end of said at least one toe spring, and an opposite end thereof molded to said heel portion of said foot plate; and
a prosthetic ankle adapted for mounting to said prosthetic foot;
wherein, when connected to said prosthetic foot, the position of said prosthetic ankle will approximate the position of a typical human ankle.

50. The low profile prosthetic foot assembly of claim 49, wherein said at least one elastomeric heel spring has at least one recess or cavity located therein, said recess or cavity affecting the compressive resistance of said at least one heel spring.

51. The low profile prosthetic foot assembly of claim 50, wherein a secondary heel spring element is located in said recess or cavity, said secondary heel spring element having a compressive modulus that differs from the compressive modulus of the elastomeric material forming said at least one heel spring.

52. The low profile prosthetic foot assembly of claim 49, further comprising a fiber reinforcement material embedded in said at least one elastomeric heel spring.

53. The low profile prosthetic foot assembly of claim 50, further comprising at least one auxiliary toe spring disposed below said at least one toe spring and connected to said at least one elastomeric heel spring.

54. The low profile prosthetic foot assembly of claim 53, further comprising a spacer plate disposed between said at least one toe spring and said at least one auxiliary toe spring.

55. A low profile prosthetic foot, comprising:
a foot plate having a heel portion and a toe portion spaced along the length thereof;
at least one primary toe spring having a distal end connected to said toe portion of said foot plate and a proximal end residing some distance above said heel portion of said foot plate;
at least one auxiliary toe spring disposed below said at least one toe spring; and
at least one heel spring residing between said heel portion of said foot plate and said proximal end of said at least one primary toe spring.

56. The low profile prosthetic foot of claim 55, wherein a pair of primary toe springs are used.

57. The low profile prosthetic foot of claim 1, wherein a pair of heel springs reside between said heel portion of said foot plate and said proximal end of said at least one toe spring.

58. The low profile prosthetic foot of claim 55, further comprising a heel attachment plate disposed between said foot plate and said at least one heel spring.

59. The low profile prosthetic foot of claim 55, further comprising a spacer plate disposed between said at least one primary toe spring and said at least one auxiliary toe spring.

60. The low profile prosthetic foot of claim 59, wherein said spacer plate includes a stop that limits the differential movement of a side of said at least one toe spring that is subjected to compression during bending thereof.

61. The low profile prosthetic foot of claim 55, wherein said at least one heel spring is a pneumatic or hydraulic shock absorber.

62. The low profile prosthetic foot of claim 55, wherein said at least one heel spring is a helical compression spring.

63. The low profile prosthetic foot of claim 55, wherein said at least one heel spring is made from an elastomeric material.

64. The low profile prosthetic foot of claim 63, wherein said at least one heel spring has at least one recess or cavity located therein, said recess or cavity affecting the compressive resistance of said at least one heel spring.

65. The low profile prosthetic foot of claim 63, wherein a secondary heel spring element is located in said recess or cavity, said secondary heel spring element having a compressive modulus that differs from the compressive modulus of the elastomeric material forming said at least one heel spring.

66. The low profile prosthetic foot of claim 63, wherein said at least one heel spring is a U-shaped element, a rounded end of which is adapted for connection to said foot plate.

67. The low profile prosthetic foot of claim 66, further comprising a threaded fastener embedded in one, or both, ends of said at least one heel spring.

68. The low profile prosthetic foot of claim 63, wherein one end of said heel spring is molded to said foot plate.

69. The low profile prosthetic foot of claim 63, wherein one end of said heel spring is molded to a proximal end of said at least one auxiliary toe spring.

70. The low profile prosthetic foot of claim 63, further comprising a fiber reinforcement material embedded in said elastomeric material.

71. The low profile prosthetic foot of claim 55, further comprising a recess in said at least one auxiliary toe spring for receiving one end of said at least one heel spring.

72. The low profile prosthetic foot of claim 55, wherein said at least one primary toe spring is secured to said at least one heel spring by passing at least one fastener through a prosthetic ankle, through said at least one primary and said at least one auxiliary toe spring, and into said at least one heel spring.

73. The low profile prosthetic foot of claim 55, wherein said distal end of said at least one primary toe spring is connected to said foot plate by a toe spring clamp assembly that is molded to said foot plate.
